(19) United States
(12) Patent Application Publication
(10) Pub. No.: US 2026/0283600 A1
(43) Pub. Date: Sep. 24, 2026

(54) ULTRASOUND DIAGNOSTIC APPARATUS AND CONTROL METHOD OF ULTRASOUND DIAGNOSTIC APPARATUS

(71) Applicant: FUJIFILM Corporation, Tokyo (JP)

(72) Inventor: Hiroyuki KARASAWA, Kanagawa (JP)

(73) Assignee: FUJIFILM Corporation, Tokyo (JP)

(21) Appl. No.: 19/561,483

(22) Filed: Mar. 9, 2026

(30) Foreign Application Priority Data
Mar. 24, 2025 (JP) 2025-048232

(51) Int. Cl. A61B 8/00

(52) U.S. Cl. CPC A61B 8/56; A61B 8/4427; A61B 8/4433; A61B 8/4472

(57) ABSTRACT

Provided are an ultrasound diagnostic apparatus and a control method of an ultrasound diagnostic apparatus that can rapidly charge an ultrasound probe while having a simple configuration.

An ultrasound diagnostic apparatus includes a mobile information terminal, an ultrasound probe that incorporates a probe battery and is wirelessly connected to the mobile information terminal, a charger that charges the probe battery, and a battery-integrated power source that incorporates a power supply battery and is connected to the charger.

### CROSS-REFERENCE TO RELATED APPLICATIONS

The present application claims priority under 35 U.S.C. § 119 to Japanese Patent Application No. 2025-048232, filed on Mar. 24, 2025. The above application is hereby expressly incorporated by reference, in its entirety, into the present application.

### BACKGROUND OF THE INVENTION

### 1. Field of the Invention

The present invention relates to an ultrasound diagnostic apparatus comprising an ultrasound probe that incorporates a battery, and a control method of the ultrasound diagnostic apparatus.

### 2. Description of the Related Art

In the related art, an ultrasound examination is performed in which an ultrasound image representing a tomographic plane inside a subject is captured using a so-called ultrasound diagnostic apparatus, and the inside of the subject is examined based on the captured ultrasound image. In order to smoothly perform such an ultrasound examination, for example, as disclosed in JP2021-129811A, an ultrasound diagnostic apparatus comprising an ultrasound probe that incorporates a battery and a mobile information terminal such as a so-called tablet that is connected to the ultrasound probe by so-called wireless communication has been developed. The battery of the ultrasound probe in JP2021-129811A is charged by surplus power of the mobile information terminal or so-called wireless power supply.

### SUMMARY OF THE INVENTION

However, in the technology of JP2021-129811A, a power supply unit for the mobile information terminal and a power supply unit for wireless power supply to the ultrasound probe are required, and the configuration of the power supply is complicated. Therefore, in a case where the power supply unit for the mobile information terminal is also used as the power supply unit for wireless power supply to the ultrasound probe, since the supply power is limited, so-called rapid wireless power supply cannot be performed, a long waiting time occurs until the ultrasound probe is sufficiently charged, and it may be difficult to smoothly perform the examination.

The present invention has been made to solve such a problem in the related art, and an object of the present invention is to provide an ultrasound diagnostic apparatus and a control method of an ultrasound diagnostic apparatus that can rapidly charge an ultrasound probe while having a simple configuration.

According to the following configuration, the above-described object can be achieved.
[1] An ultrasound diagnostic apparatus comprising:
a mobile information terminal;
an ultrasound probe that incorporates a probe battery and is wirelessly connected to the mobile information terminal;
a charger that charges the probe battery; and
a battery-integrated power source that incorporates a power supply battery and is connected to the charger,
in which the ultrasound probe is charged by the charger using power supplied from the battery-integrated power source.
[2] The ultrasound diagnostic apparatus according to [1], in which the battery-integrated power source supplies an output power of 7.5 W or more and 240 W or less to the charger.
[3] The ultrasound diagnostic apparatus according to [1] or [2], in which the battery-integrated power source supplies a voltage and a current selected according to a specification of the charger among a plurality of predetermined voltages and currents to the charger.
[4] The ultrasound diagnostic apparatus according to [3], in which the battery-integrated power source includes a communication unit that receives information from the charger, and supplies to the charger a voltage and a current selected in accordance with a specification of the charger received via the communication unit from the charger.
[5] The ultrasound diagnostic apparatus according to any one of [1] to [4], in which the power supply battery of the battery-integrated power source is charged by the mobile information terminal.
[6] The ultrasound diagnostic apparatus according to [5], in which the battery-integrated power source supplies to the charger an output power larger than input power input from the mobile information terminal.
[7] The ultrasound diagnostic apparatus according to [5] or [6], further comprising: an external power source that is connected to the mobile information terminal.
[8] The ultrasound diagnostic apparatus according to any one of [1] to [4], further comprising:
an external power source that is connected to the battery-integrated power source,
in which the power supply battery of the battery-integrated power source is charged by the external power source.
[9] The ultrasound diagnostic apparatus according to [8], in which the battery-integrated power source supplies power to the mobile information terminal.
[10] The ultrasound diagnostic apparatus according to any one of [1] to [9], further comprising:
a probe holder that holds the ultrasound probe and in which the charger is disposed.
[11] The ultrasound diagnostic apparatus according to [10], further comprising:
a terminal holder that holds the mobile information terminal;
a probe holder; and
a holding stand that includes the battery-integrated power source.
[12] The ultrasound diagnostic apparatus according to any one of [1] to [11], in which the charger is a wireless charger that wirelessly charges the probe battery.
[13] A control method of an ultrasound diagnostic apparatus in which an ultrasound probe that incorporates a probe battery is wirelessly connected to a mobile information terminal, the control method comprising:
connecting a battery-integrated power source that incorporates a power supply battery to a charger; and
charging the ultrasound probe by the charger using power supplied from the battery-integrated power source.

In the ultrasound diagnostic apparatus according to the present invention, since the ultrasound diagnostic apparatus comprises the mobile information terminal, the ultrasound probe that incorporates the probe battery and is wirelessly connected to the mobile information terminal, the charger that charges the probe battery, and the battery-integrated power source that incorporates the power supply battery and is connected to the charger, the ultrasound probe can be rapidly charged while having a simple configuration.

### BRIEF DESCRIPTION OF THE DRAWINGS

FIG. 1 is a block diagram showing a configuration of an ultrasound diagnostic apparatus according to Embodiment 1 of the present invention.

FIG. 2 is a block diagram showing an internal configuration of an ultrasound probe in Embodiment 1 of the present invention.

FIG. 3 is a block diagram showing an internal configuration of a transmission/reception circuit in Embodiment 1 of the present invention.

FIG. 4 is a block diagram showing an internal configuration of an image generation unit in Embodiment 1 of the present invention.

FIG. 5 is a diagram showing an example of a terminal holder that holds the mobile information terminal and a probe holder that holds the ultrasound probe according to Embodiment 1 of the present invention.

FIG. 6 is a diagram showing an enlarged view of the probe holder according to Embodiment 1 of the present invention.

FIG. 7 is a block diagram showing an internal configuration of a battery-integrated power source according to a modification example of Embodiment 1 of the present invention.

FIG. 8 is a block diagram showing a configuration of an ultrasound diagnostic apparatus according to Embodiment 2 of the present invention.

FIG. 9 is a block diagram showing a configuration of an ultrasound diagnostic apparatus according to Embodiment 3 of the present invention.

### DESCRIPTION OF THE PREFERRED EMBODIMENTS

Hereinafter, embodiments of the invention will be described with reference to the accompanying drawings.

The description of components described below is provided based on a representative embodiment of the present invention, but the present invention is not limited to such an embodiment.

In the present specification, a numerical range represented using “to” means a range including numerical values before and after “to” as a lower limit value and an upper limit value.

In the present specification, the terms “same” and “identical” include an error range generally allowed in the technical field.

### Embodiment 1

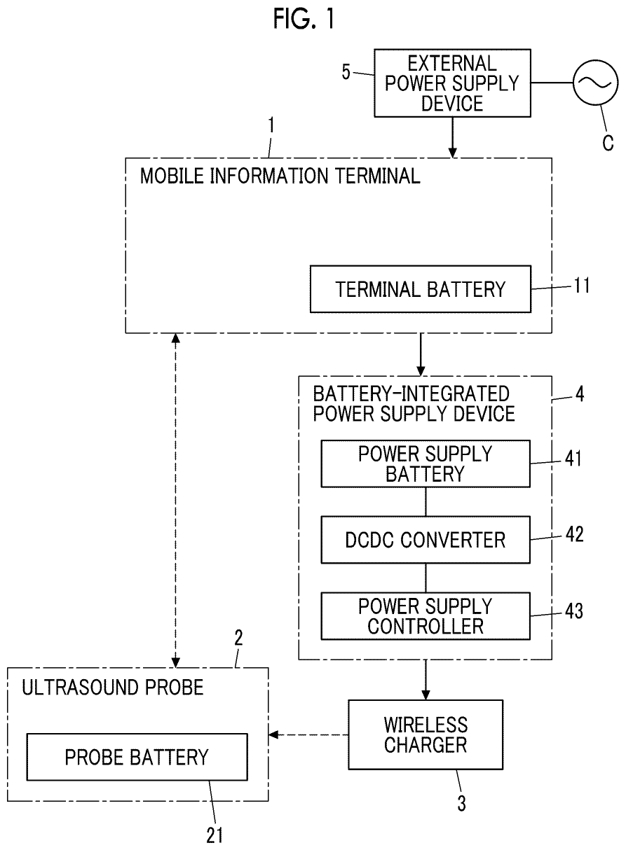

FIG. 1 shows a configuration of an ultrasound diagnostic apparatus according to Embodiment 1 of the present invention. The ultrasound diagnostic apparatus comprises a mobile information terminal 1, an ultrasound probe 2 that is wirelessly connected to the mobile information terminal 1, a wireless charger 3 that charges the ultrasound probe 2, a battery-integrated power source 4 that is connected to the wireless charger 3, and an external power source 5 that is connected to the mobile information terminal 1. The external power source 5 is configured by a so-called alternating current adapter (AC adapter) or the like, and is connected to an alternating current power supply C having a specified voltage value.

The mobile information terminal 1 is a handheld information terminal called a so-called tablet or smartphone, and comprises a terminal battery 11 and a monitor (not shown). The mobile information terminal 1 is used to display an ultrasound image captured by the ultrasound probe 2 or the like as described below.

The mobile information terminal 1 is driven by power supplied from the terminal battery 11. The terminal battery 11 is charged by the external power source 5 connected to the alternating current power supply C, and supplies power to the battery-integrated power source 4. The power supplied from the terminal battery 11 to the battery-integrated power source 4 is often limited to, for example, 4.5 W by a standard of the tablet or the like constituting the mobile information terminal 1. Various batteries can be used as the terminal battery 11, but for example, a so-called lithium-ion battery can be used.

The ultrasound probe 2 is wirelessly connected to the mobile information terminal 1, and transmits and receives ultrasonic waves to and from the inside of the subject to capture an ultrasound image. The ultrasound probe 2 incorporates a probe battery 21. The internal configuration of the ultrasound probe 2 will be described below.

The wireless charger 3 wirelessly supplies the power supplied from the battery-integrated power source 4 to the ultrasound probe 2 by a so-called electromagnetic induction method. The wireless charger 3 includes a power transmitting coil described below, and transmits a search signal for searching for a power receiving coil of the ultrasound probe 2 described below to the ultrasound probe 2. The wireless charger 3 detects that the ultrasound probe 2 is positioned to be chargeable by the wireless charger 3 by receiving a response signal from the power receiving coil that has received the search signal. The wireless charger 3 transmits the detection signal to the battery-integrated power source 4.

The battery-integrated power source 4 includes a power supply battery 41, a direct current/direct current (DCDC) converter 42 connected to the power supply battery 41, and a power supply controller 43 connected to the DCDC converter 42.

The power supply battery 41 is a battery used for power supply to the ultrasound probe 2 via the wireless charger 3. The power supply battery 41 is charged by being supplied with power from the terminal battery 11 of the mobile information terminal 1. Various batteries can be used as the power supply battery 41, but for example, a so-called lithium-ion battery can be used.

The DCDC converter 42 converts a voltage applied from the power supply battery 41 into a higher voltage or a lower voltage. For example, a power value of 4.5 W input to the battery-integrated power source 4 can be boosted to an output power of 7.5 W or more and 240 W or less. As a result, the battery-integrated power source 4 can supply the wireless charger 3 with an output power larger than the input power input from the mobile information terminal 1, and rapidly charge the ultrasound probe 2 via the wireless charger 3.

Here, as an example, it is considered that, for example, the ultrasound probe 2 having a power consumption of 12.6 W in the ultrasound scanning and the ultrasound probe 2 having a power consumption of 3.8 W in the ultrasound scanning are used for 10 minutes, and then the probe battery 21 of each ultrasound probe 2 is charged to a remaining amount before use. In a case of considering charging with a charger of a so-called universal serial bus power delivery (USB PD) standard and a wireless charging charger, in general, a charging efficiency in the USB PD standard is 80%, and a charging efficiency in the wireless charging is 50%. The supply power of the charger required in a case of charging the probe battery 21 of each ultrasound probe 2 to the remaining amount before use for 10.0 minutes, 5.0 minutes, 2.5 minutes, and 1.0 minutes is as shown in Table 1. From this table, it can be seen that, in a case where the output power of the battery-integrated power source 4 is set to 7.5 W or more and 240 W or less, the probe battery 21 of the ultrasound probe 2 can be sufficiently rapidly charged.

TABLE 1

| Power | Charging | Charging | Charging time | | | |
|---|---|---|---|---|---|---|
| consumption | method | efficiency | 10.0 minutes | 5.0 minutes | 2.5 minutes | 1.0 minute |
| 12.6 W | USB PD | 80% | 15.8 W | 31.5 W | 63.0 W | 157.5 W |
| | Wireless | 50% | 25.2 W | 50.4 W | 100.8 W | 252.0 W |
| | charging | | | | | |
| 3.8 W | USB PD | 80% | 4.8 W | 9.5 W | 19.0 W | 47.5 W |
| | Wireless | 50% | 7.6 W | 15.2 W | 30.4 W | 76.0 W |
| | charging | | | | | |

The power supply controller 43 controls the power supply from the power supply battery 41 via the DCDC converter 42 to the wireless charger 3. The power supply controller 43 can supply power to the wireless charger 3 in a case of receiving a detection signal indicating that the power receiving coil of the ultrasound probe 2 is detected from the wireless charger 3, and can stop the power supply to the wireless charger 3 in a case of being unable to receive the detection signal from the wireless charger 3.

The external power source 5 is an AC adapter, and is reinforced with insulation in order to satisfy standards such as a so-called means of operator protection (MOOP) and a so-called means of patient protection (MOPP) in IEC 60601-1, which is a standard related to a medical device.

As described above, in the ultrasound diagnostic apparatus according to Embodiment 1, the battery-integrated power source 4 is connected to the mobile information terminal 1 connected to the external power source 5, and the wireless charger 3 for supplying power to the ultrasound probe 2 is connected to the battery-integrated power source 4. Therefore, for example, it is not necessary to connect different power sources to the mobile information terminal 1 and the wireless charger 3, respectively, and the ultrasound diagnostic apparatus can have a simple configuration. In addition, by connecting the battery-integrated power source 4 to the wireless charger 3, for example, a large amount of power can be supplied to the ultrasound probe 2 via the wireless charger 3 to rapidly charge the probe battery 21 of the ultrasound probe 2, as compared to a case where the mobile information terminal 1 is directly connected to the wireless charger 3. In addition, for example, even in a case where the power remaining amount of the terminal battery 11 of the mobile information terminal 1 is significantly low or zero, the probe battery 21 of the ultrasound probe 2 can be charged by the battery-integrated power source 4 having the power supply battery 41.

In addition, for example, in a case where the ultrasound diagnostic apparatus has a plurality of power sources, it is necessary to perform the reinforced insulation for satisfying the standard such as IEC 60601-1 for each power source. However, since the ultrasound diagnostic apparatus comprises only one external power source 5, only one external power source 5 subjected to the reinforced insulation may be provided, and the manufacturing cost can be suppressed.

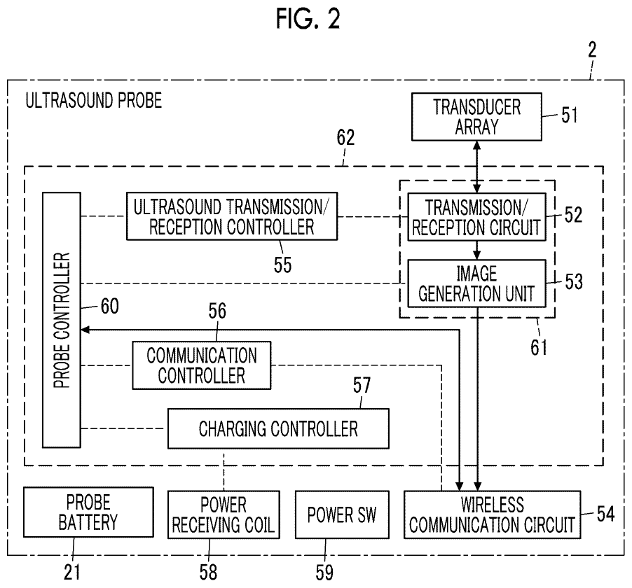

Here, the internal configuration of the ultrasound probe 2 will be described. As shown in FIG. 2, the ultrasound probe 2 comprises a transducer array 51 in addition to the probe battery 21. A transmission/reception circuit 52, an image generation unit 53, and a wireless communication circuit 54 are sequentially connected to the transducer array 51 . . . . An ultrasound transmission/reception controller 55 is connected to the transmission/reception circuit 52. A communication controller 56 is connected to the wireless communication circuit 54. The ultrasound probe 2 comprises a charging controller 57, and a power receiving coil 58 is connected to the charging controller 57. In addition, the ultrasound probe 2 comprises a power switch 59. In addition, a probe controller 60 is connected to the image generation unit 53, the wireless communication circuit 54, the ultrasound transmission/reception controller 55, the communication controller 56, and the charging controller 57. The transmission/reception circuit 52 and the image generation unit 53 constitute an image acquisition unit 61. In addition, a processor 62 for the ultrasound probe 2 is configured by the transmission/reception circuit 52, the image generation unit 53, the ultrasound transmission/reception controller 55, the communication controller 56, the charging controller 57, and the probe controller 60.

The transducer array 51 of the ultrasound probe 2 includes a plurality of ultrasound transducers arranged in a one-dimensional or two-dimensional manner. In accordance with a drive signal supplied from the transmission/reception circuit 52, each of the ultrasound transducers transmits ultrasound and receives an ultrasound echo from a subject to output a signal based on the ultrasound echo. Each ultrasound transducer is configured by, for example, forming electrodes at both ends of a piezoelectric material consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), and the like.

The image acquisition unit 61 configured by the transmission/reception circuit 52 and the image generation unit 53 acquires an ultrasound image in which a tomographic plane inside the subject is captured by transmitting and receiving the ultrasound beam using the ultrasound probe 2.

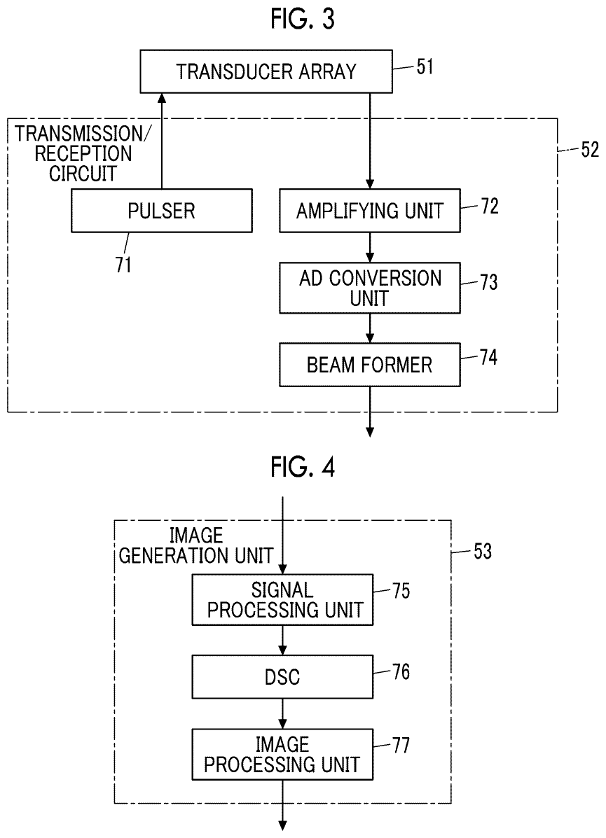

The transmission/reception circuit 52 transmits the ultrasound wave from the transducer array 51 and generates a sound ray signal based on a reception signal acquired by the transducer array 51, under the control of the probe controller 60. As shown in FIG. 3, the transmission/reception circuit 52 includes a pulser 71 connected to the transducer array 51, and an amplifying unit 72, an analog-to-digital (AD) conversion unit 73, and a beam former 74 that are sequentially connected in series to the transducer array 51.

The pulser 71 includes, for example, a plurality of pulse generators, and adjusts an amount of delay of each of drive signals and supplies the drive signals to the plurality of ultrasound transducers such that ultrasound waves transmitted from the plurality of ultrasound transducers of the transducer array 51 form an ultrasound beam based on a transmission delay pattern selected according to a control signal from the ultrasound transmission/reception controller 55 and the probe controller 60. As described above, in a case in which a pulsed or continuous wave-like voltage is applied to the electrodes of the ultrasound transducer of the transducer array 51, the piezoelectric material expands and contracts to generate pulsed or continuous wave-like ultrasound from each of the ultrasound transducers, whereby the ultrasound beam is formed from the combined wave of the ultrasound.

The transmitted ultrasound beam is, for example, reflected by a target such as a part of the subject and propagates toward the transducer array 51 of the ultrasound probe 2. The ultrasound echo propagating toward the transducer array 51 in this way is received by each of the ultrasound transducers constituting the transducer array 51. In such a case, each of the ultrasound transducers constituting the transducer array 51 receives the propagating ultrasound echo to expand and contract, generates the reception signal, which is an electrical signal, and outputs these reception signals to the amplifying unit 72.

The amplifying unit 72 amplifies the signal input from each of the ultrasound transducers constituting the transducer array 51 and transmits the amplified signal to the AD conversion unit 73. The AD conversion unit 73 converts the signal transmitted from the amplifying unit 72 into digital reception data. The beam former 74 performs so-called reception focus processing by applying and adding the delay to each reception data received from the AD conversion unit 73. By the reception focus processing, each reception data, which is converted by the AD conversion unit 73, is phase-added, and the sound ray signal in which the focus of the ultrasound echo is narrowed down is acquired.

As shown in FIG. 4, the image generation unit 53 has a configuration in which a signal processing unit 75, a digital scan converter (DSC) 76, and an image processing unit 77 are sequentially connected in series.

The signal processing unit 75 generates a B-mode image signal, which is tomographic image information regarding tissues inside the subject, by performing, on the sound ray signal received from the transmission/reception circuit 52, correction of the attenuation due to the distance according to the depth of the reflection position of the ultrasound wave using a sound velocity value set by the probe controller 60 and then performing envelope detection processing.

The DSC 76 converts (raster-converts) the B-mode image signal, which is generated by the signal processing unit 75, into the image signal in accordance with a normal television signal scanning method.

The image processing unit 77 performs various necessary image processing such as gradation processing on the B-mode image signal input from the DSC 76, and then transmits the B-mode image signal to the wireless communication circuit 54. The B-mode image signal subjected to the image processing by the image processing unit 77 is referred to as an ultrasound image.

The ultrasound transmission/reception controller 55 controls the transmission/reception circuit 52 to transmit and receive the ultrasonic waves according to a transmission/reception condition determined in the transducer array 51. The determined transmission/reception condition can include, for example, a transmission pattern of the ultrasonic waves, a transmission focus position of the ultrasonic waves, a reception focus position, a display depth of the ultrasound image, and conditions of the gain and the dynamic range of the reception signal.

The wireless communication circuit 54 is a circuit that wirelessly transmits the ultrasound image acquired by the image acquisition unit 61 to the mobile information terminal 1 and wirelessly transmits and receives other necessary information to and from the mobile information terminal 1.

The communication controller 56 controls the transmission and reception of information to and from the mobile information terminal 1 by the wireless communication circuit 54. The communication controller 56 can cause the wireless communication circuit 54 to transmit and receive, for example, information for the ultrasound probe 2 to wirelessly connect to the mobile information terminal 1, and transmit the ultrasound image to the mobile information terminal 1.

The power receiving coil 58 receives the power supplied from the wireless charger 3 by sensing the magnetic field emitted from the wireless charger 3. In addition, the power receiving coil 58 emits the response signal in response to the search signal transmitted from the wireless charger 3.

The charging controller 57 controls the charging of the probe battery 21 by controlling the operation of the power receiving coil 58.

The power switch (power SW) 59 is a switch that turns on or off the power supply state of the ultrasound probe 2. The power switch 59 is operated by, for example, a user.

In the present embodiment, each processing in the processor 62 is executed by any computer. Moreover, any computer may execute these processes by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the above-described order and may be changed as appropriate. Any computer may be a general-purpose computer, a computer for specific use, a workstation, or another system capable of executing each processing.

The processor 62 may be composed of one or a plurality of pieces of hardware, and types of hardware are not limited. For example, the processor 62 may be configured with hardware such as a central processing unit (CPU), a micro processing unit (MPU), a programmable logic device such as a field-programmable gate array (FPGA), a dedicated circuit for executing specific processing such as an application-specific integrated circuit (ASIC), a graphic processing unit (GPU), or a neural processing unit (NPU). Additionally, the types of hardware may be a combination of different types of hardware. In a case where the plurality of types of hardware are configured to execute one or a plurality of types of processing of a certain processor, the plurality of types of hardware may exist in devices physically separated from each other or may exist in the same device. In addition, in any of the embodiments, the order of each processing by the processor 62 is not limited to the above order and may be changed as appropriate. The hardware is composed of an electric circuit (circuitry) in which circuit elements such as semiconductor elements are combined.

The program may be software such as firmware or a microcode. Furthermore, the program may be, for example, a program module group, and each function thereof may be implemented by a processor configured to execute each function. The program may be a program code or a plurality of code segments stored in one or a plurality of non-transitory computer-readable media (for example, a storage medium and other storages). The program may be stored in the plurality of non-transitory computer-readable media existing in physically separated devices. The program code or the code segment may represent any combination of procedures, functions, subprograms, routines, subroutines, modules, software packages, classes, instructions, data structures, or program statements. The program code or the code segments may be connected to other code segments or hardware circuits by transmitting and receiving information, data, an argument, a parameter, or content of a memory.

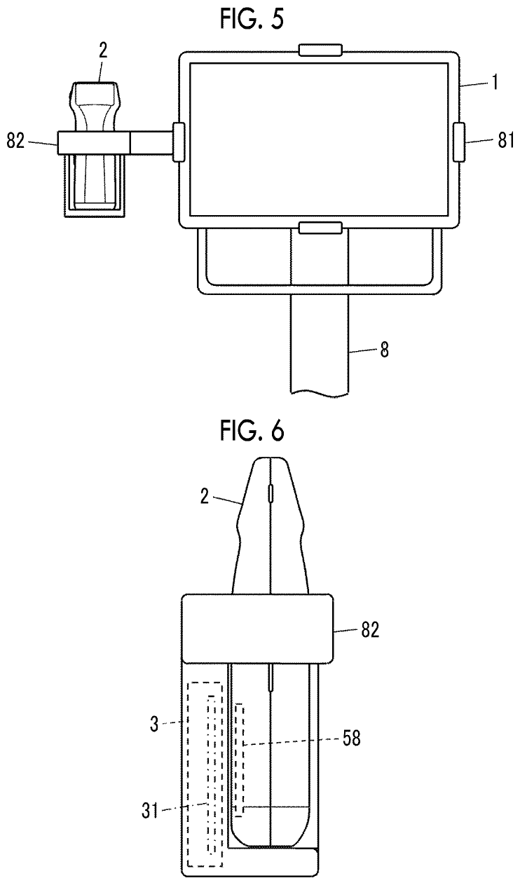

Next, an example of an appearance of the ultrasound diagnostic apparatus will be described. As shown in FIG. 5, the ultrasound diagnostic apparatus can comprise a terminal holder 81 that holds the mobile information terminal 1, a probe holder 82 that holds the ultrasound probe 2, and a holding stand 8 including the battery-integrated power source 4. The probe holder 82 holds the ultrasound probe 2 in a standing state, for example.

As shown in FIG. 6, the probe holder 82 can incorporate the wireless charger 3. The wireless charger 3 includes a power transmitting coil 31 that emits a magnetic field for supplying power to the ultrasound probe 2, and the power transmitting coil 31 and the power receiving coil 58 of the ultrasound probe 2 are disposed to face each other in a state where the ultrasound probe 2 is held by the probe holder 82. The wireless charger 3 is electrically connected to the battery-integrated power source 4 via a wiring line disposed in the holding stand 8, and in a case where the ultrasound probe 2 is held by the probe holder 82, the power is supplied from the battery-integrated power source 4 to the ultrasound probe 2 via the wireless charger 3.

Since the ultrasound probe 2 is rapidly charged by the battery-integrated power source 4, for example, even in a case where the power remaining amount of the probe battery 21 is reduced and the ultrasound examination cannot be performed, the user does not have to wait for a long time until the probe battery 21 is sufficiently charged to perform the ultrasound examination.

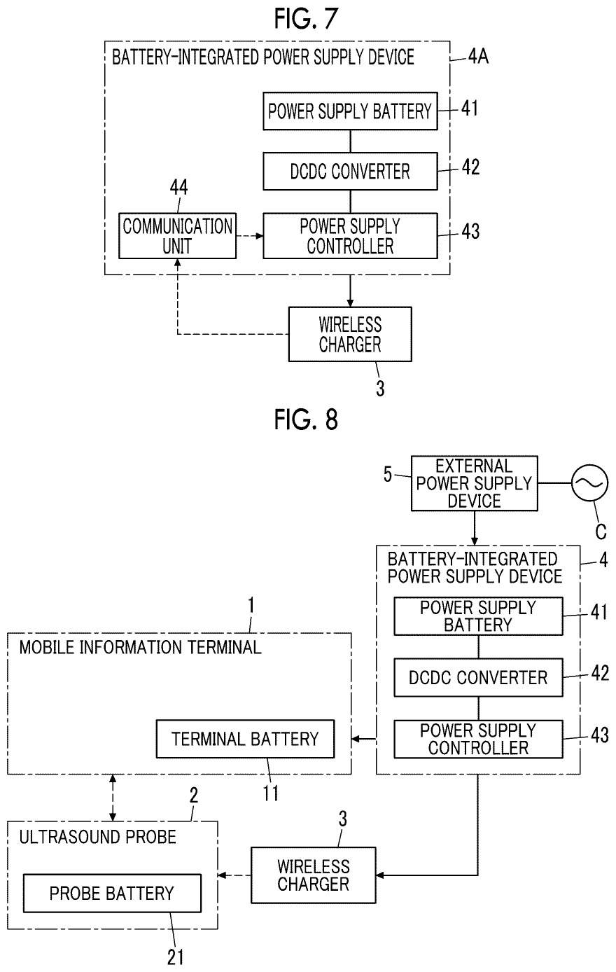

In general, the wireless charger 3 may have a voltage or a current that can be supplied to a power supply target device defined by a specification. Therefore, the battery-integrated power source 4 can also supply the voltage and the current selected according to the specification of the wireless charger 3 to the wireless charger 3. In this case, the ultrasound diagnostic apparatus can comprise, for example, a battery-integrated power source 4A shown in FIG. 7. The battery-integrated power source 4A further comprises a communication unit 44 in the battery-integrated power source 4 shown in FIG. 1. The communication unit 44 is connected to the wireless charger 3 and the power supply controller 43.

The communication unit 44 receives information on the specification of the wireless charger 3 from the wireless charger 3, and transmits the information to the power supply controller 43.

The power supply controller 43 stores the specifications of the plurality of wireless chargers 3 and predetermined values of the voltage and the current corresponding to the specifications in advance, and selects the predetermined voltage and current from among the plurality of predetermined voltages and currents according to the specification of the wireless charger 3 received from the wireless charger 3 via the communication unit 44. The power supply controller 43 further supplies the selected voltage and current to the wireless charger 3.

As a result, for example, even in a case where the currently used wireless charger 3 is replaced with another wireless charger 3 having a different specification, the voltage and the current corresponding to the specification of the replaced wireless charger 3 can be supplied to the wireless charger 3.

In addition, although the ultrasound probe 2 comprises the image generation unit 53, for example, the mobile information terminal 1 can comprise the image generation unit 53 instead of the ultrasound probe 2 comprising the image generation unit 53. In this case, the ultrasound signal is transmitted from the transmission/reception circuit 52 to the mobile information terminal 1 via the wireless communication circuit 54. The image generation unit 53 of the mobile information terminal 1 processes the sound ray signal received from the ultrasound probe 2 to generate the ultrasound image.

In addition, in Embodiment 1, the ultrasound diagnostic apparatus comprises the wireless charger 3, and the probe battery 21 is charged by the wireless charger 3. However, the ultrasound diagnostic apparatus can comprise, for example, a wired charger having a USB PD standard instead of the wireless charger 3. The wired charger includes a so-called USB connector. Even in this case, the probe battery 21 can be rapidly charged from the battery-integrated power source 4.

### Embodiment 2

Although an example has been described in which the battery-integrated power source 4 supplies power to only the ultrasound probe 2 via the wireless charger 3, the battery-integrated power source 4 can further supply power to the mobile information terminal 1.

FIG. 8 shows a configuration of an ultrasound diagnostic apparatus of Embodiment 2. The ultrasound diagnostic apparatus according to Embodiment 2 comprises the mobile information terminal 1, the ultrasound probe 2, the wireless charger 3, the battery-integrated power source 4, and the external power source 5 connected to the alternating current power supply C, as in the ultrasound diagnostic apparatus according to Embodiment 1 shown in FIG. 1, but the battery-integrated power source 4 is connected to the external power source 5, and the mobile information terminal 1 and the wireless charger 3 are connected to the battery-integrated power source 4.

The power supply battery 41 of the battery-integrated power source 4 is charged by the external power source 5. In addition, the battery-integrated power source 4 supplies power to the mobile information terminal 1 in addition to the wireless charger 3.

As described above, even in a case where the battery-integrated power source 4 supplies power to the mobile information terminal 1 and the wireless charger 3, for example, it is not necessary to connect different power sources to the mobile information terminal 1 and the wireless charger 3, respectively, and the ultrasound diagnostic apparatus can have a simple configuration. In addition, in this case, not only the ultrasound probe 2 but also the mobile information terminal 1 can be rapidly charged by the battery-integrated power source 4.

In Embodiment 2, the ultrasound diagnostic apparatus comprises the wireless charger 3, and the probe battery 21 is charged by the wireless charger 3. However, the ultrasound diagnostic apparatus can comprise, for example, a wired charger having a USB PD standard instead of the wireless charger 3. Even in this case, the probe battery 21 can be rapidly charged from the battery-integrated power source 4.

### Embodiment 3

In Embodiment 1 and Embodiment 2, the ultrasound diagnostic apparatus comprises one wireless charger 3, but can comprise a plurality of wireless chargers 3.

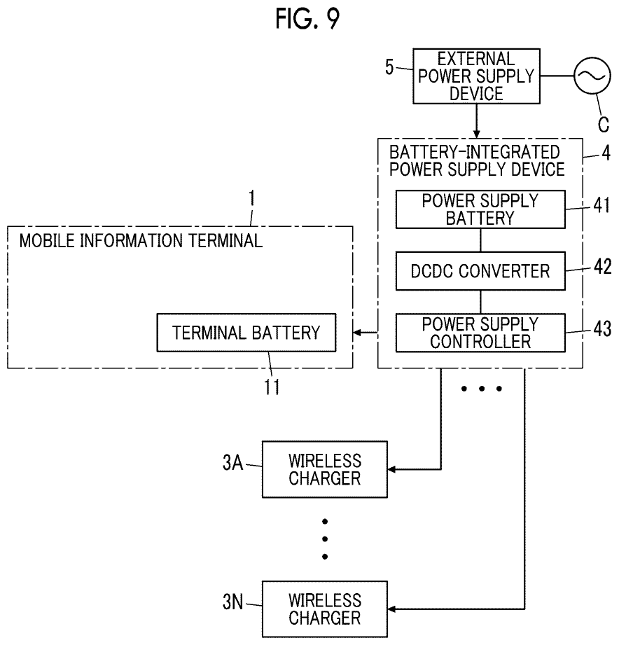

FIG. 9 shows a configuration of an ultrasound diagnostic apparatus of Embodiment 3. The ultrasound diagnostic apparatus according to Embodiment 3 comprises N wireless chargers 3A to 3N instead of one wireless charger 3 in the ultrasound diagnostic apparatus according to Embodiment 2 shown in FIG. 8. Here, N denotes the number that is two or more. The N wireless chargers 3A to 3N supply power to the N ultrasound probes 2. In FIG. 9, the N ultrasound probes 2 are omitted for description.

As described above, by comprising the plurality of wireless chargers 3A to 3N, the ultrasound diagnostic apparatus can simultaneously rapidly charge the plurality of ultrasound probes 2. In the ultrasound examination of the subject, for example, even in a case where a plurality of types of ultrasound probes 2 such as a so-called linear type ultrasound probe 2 and a so-called convex type ultrasound probe 2 are used, the user does not have to wait for a long time until the probe battery 21 of the plurality of ultrasound probes 2 is sufficiently charged to perform the ultrasound examination.

In Embodiment 3, the ultrasound diagnostic apparatus comprises the plurality of wireless chargers 3A to 3N, and the probe battery 21 is charged by the plurality of wireless chargers 3A to 3N. However, the ultrasound diagnostic apparatus can comprise, for example, a plurality of wired chargers having a USB PD standard instead of the plurality of wireless chargers 3A to 3N. Even in this case, the probe battery 21 can be rapidly charged from the battery-integrated power source 4.

### EXPLANATION OF REFERENCES

1: mobile information terminal
2: ultrasound probe
3, 3A, 3N: wireless charger
4, 4A: battery-integrated power source
5: external power source
8: holding stand
11: terminal battery
21: probe battery
31: power transmitting coil
41: power supply battery
42: DCDC converter
43: power supply controller
44: communication unit
51: transducer array
52: transmission/reception circuit
53: image generation unit
54: wireless communication circuit
55: ultrasound transmission/reception controller
56: communication controller
57: charging controller
58: power receiving coil
59: power switch
60: probe controller
61: image acquisition unit
62: processor
71: pulser
72: amplifying unit
73: AD conversion unit
74: beam former
75: signal processing unit
76: DSC
77: image processing unit
81: terminal holder
82: probe holder

What is claimed is:

1. An ultrasound diagnostic apparatus comprising:
a mobile information terminal;
an ultrasound probe that incorporates a first battery and is wirelessly connected to the mobile information terminal;
a charger configured to charge the first battery; and
a battery-integrated power source that incorporates a second battery and is connected to the charger,
wherein the ultrasound probe is charged by the charger using power supplied from the battery-integrated power source.

2. The ultrasound diagnostic apparatus according to claim 1,
wherein the battery-integrated power source is configured to supply an output power of 7.5 W or more and 240 W or less to the charger.

3. The ultrasound diagnostic apparatus according to claim 1,
wherein the battery-integrated power source is configured to supply a voltage and a current selected according to a specification of the charger among a plurality of predetermined voltages and currents to the charger.

4. The ultrasound diagnostic apparatus according to claim 2,
wherein the battery-integrated power source is configured to supply a voltage and a current selected according to a specification of the charger among a plurality of predetermined voltages and currents to the charger.

5. The ultrasound diagnostic apparatus according to claim 3,
wherein the battery-integrated power source is configured to
receive information of a specification of the charger from the charger; and
supply to the charger a voltage and a current selected in accordance with the information of the specification of the charger.

6. The ultrasound diagnostic apparatus according to claim 1,
wherein the second battery of the battery-integrated power source is charged by the mobile information terminal.

7. The ultrasound diagnostic apparatus according to claim 2,
wherein the second battery of the battery-integrated power source is charged by the mobile information terminal.

8. The ultrasound diagnostic apparatus according to claim 3,
wherein the second battery of the battery-integrated power source is charged by the mobile information terminal.

9. The ultrasound diagnostic apparatus according to claim 4,
wherein the second battery of the battery-integrated power source is charged by the mobile information terminal.

10. The ultrasound diagnostic apparatus according to claim 6,
wherein the battery-integrated power source is configured to supply to the charger an output power larger than input power input from the mobile information terminal.

11. The ultrasound diagnostic apparatus according to claim 6, further comprising:
an external power source that is connected to the mobile information terminal.

12. The ultrasound diagnostic apparatus according to claim 10, further comprising:
an external power source that is connected to the mobile information terminal.

13. The ultrasound diagnostic apparatus according to claim 1, further comprising:
an external power source that is connected to the battery-integrated power source,
wherein the second battery of the battery-integrated power source is charged by the external power source.

14. The ultrasound diagnostic apparatus according to claim 2, further comprising:
an external power source that is connected to the battery-integrated power source,
wherein the second battery of the battery-integrated power source is charged by the external power source.

15. The ultrasound diagnostic apparatus according to claim 3, further comprising:
an external power source that is connected to the battery-integrated power source,
wherein the second battery of the battery-integrated power source is charged by the external power source.

16. The ultrasound diagnostic apparatus according to claim 13,
wherein the battery-integrated power source is configured to supply power to the mobile information terminal.

17. The ultrasound diagnostic apparatus according to claim 1, further comprising:
a probe holder that holds the ultrasound probe and in which the charger is disposed.

18. The ultrasound diagnostic apparatus according to claim 17, further comprising:
a terminal holder that holds the mobile information terminal;
a probe holder; and
a holding stand that includes the battery-integrated power source.

19. The ultrasound diagnostic apparatus according to claim 1,
wherein the charger is a wireless charger configured to charge the first battery.

20. A control method of an ultrasound diagnostic apparatus in which an ultrasound probe that incorporates a first battery is wirelessly connected to a mobile information terminal, the control method comprising:
connecting a battery-integrated power source that incorporates a second battery to a charger; and
charging the ultrasound probe by the charger using power supplied from the battery-integrated power source.
